(19) United States
(12) Patent Application Publication
(10) Pub. No.: US 2024/0261149 A1
(43) Pub. Date: Aug. 8, 2024

(54) METHOD FOR DETERMINING AN OPTIMIZED SPATIAL PULSE DISTANCE OF LASER PULSES FOR AN OPHTHALMOLOGICAL LASER

(71) Applicant: SCHWIND EYE-TECH-SOLUTIONS GMBH, Kleinostheim (DE)

(72) Inventors: Samuel ARBA MOSQUERA, Aschaffenburg (DE); Pascal NAUBEREIT, Laufach (DE)

(21) Appl. No.: 18/436,918

(22) Filed: Feb. 8, 2024

(30) Foreign Application Priority Data
Feb. 8, 2023 (DE) 10 2023 103 038.8

(51) Int. Cl. A61F 9/008; A61B 17/00

(52) U.S. Cl. CPC A61F 9/00804; A61F 9/00827; A61B 2017/00194; A61F 2009/00872

(57) ABSTRACT

The invention relates to a method for determining an optimized spatial pulse distance of laser pulses for an ophthalmological laser of a treatment apparatus. The method includes determining, by a control device of the treatment apparatus, a laser pulse effect diameter based on a predetermined tissue factor of tissue to be irradiated and a laser pulse energy portion above an optical breakthrough threshold. The method further includes determining the optimized spatial pulse distance based on the determined laser pulse effect diameter and a preset overlap factor for adjacent laser pulses.

### FIELD

The invention relates to a method for determining an optimized spatial pulse distance of laser pulses for an ophthalmological laser of a treatment apparatus. Furthermore, the invention relates to a control device, which is formed to perform the method; to a treatment apparatus with an ophthalmological laser and such a control device; to a computer program including commands, which cause the treatment apparatus to execute the method; and to a computer-readable medium, on which the computer program is stored.

### BACKGROUND

Treatment apparatuses and methods for controlling ophthalmological lasers for correcting an optical visual disorder and/or pathologically or unnaturally altered areas of the cornea are known in the prior art. Therein, pulsed lasers and a beam focusing device can for example be formed such that laser pulses effect an optical breakthrough, in particular a photodisruption and/or photoablation, in a focus situated within the organic tissue, to remove a tissue, in particular a tissue lenticule, from the cornea. Alternatively or additionally, ophthalmological lasers can be operated below an optical breakthrough threshold to achieve a characteristic change of the cornea, in particular a laser-induced refractive index change (LIRIC) or a crosslinking.

### SUMMARY

By focusing a laser pulse, non-linear absorption processes arise within the focus volume, which results in a very fast temperature and pressure increase in the form of a laser-induced optical breakthrough in the form of a plasma expansion upon exceeding a critical value. Herein, a shock wave arises, which propagates into the surrounding medium and causes the formation of a cavitation bubble, which allows the separation of the tissue. The high temperature and the high pressure of the gas in the bubble then result in the bubble oscillation, wherein this image applies to water and cannot be completely transferred to corneal tissue, in which the bubble expansion is limited against the restoring forces of the lamellar structure of the cornea. This has severe influences on intrastromal bubble dynamics, which finally results in a smaller bubble size in the cornea compared to water with identical pulse energy. Accordingly, an incision efficiency of the laser can be reduced and not be in an optimum range.

It is the object of the present invention to optimize spatial pulse distances of laser pulses for an ophthalmological laser.

This object is solved by the independent claims. Advantageous embodiments are disclosed in the dependent claims, in the following description as well as the figures.

The invention is based on the idea that a laser pulse effect diameter, thus for example a diameter of a generated cavitation bubble, on which an energy portion depends, which is above a threshold for a laser-induced optical breakthrough. Hereby, the pulse distances of adjacent laser pulses and/or adjacent laser pulse paths can then be adjusted to generate contiguous incisions. Alternatively, energies below the optical breakthrough location can also be used to for example ascertain suitable distances of a laser-induced refractive index change.

An aspect of the invention relates to a method for determining an optimized spatial pulse distance of laser pulses for an ophthalmological laser of a treatment apparatus, wherein a laser pulse effect diameter is ascertained by a control device of the treatment apparatus, wherein the laser pulse effect diameter is ascertained depending on a predetermined tissue factor of the tissue to be irradiated and a laser pulse energy portion above an optical breakthrough threshold, and wherein the spatial pulse distance is determined from the ascertained laser pulse effect diameter and a preset overlap factor for adjacent laser pulses.

In other words, control data for the laser and/or the treatment apparatus can be ascertained, by which a beam deflection device of the treatment apparatus can for example be controlled such that a placement of laser pulses in an irradiation pattern is planned according to the spatial pulse distance, which has been ascertained by the method. This means that the laser can then be controlled with the control data and/or the ascertained spatial pulse distance. Therein, the laser pulse effect diameter is the diameter of the effect, which the laser pulse causes in the tissue, for example a diameter of a generated cavitation bubble in a cornea. Therein, this laser pulse effect diameter can depend on a laser pulse energy portion above a preset or predetermined optical breakthrough threshold on the one hand and on a tissue factor, in particular a tissue factor of a cornea, on the other hand. For example, the tissue factor can be predetermined by measurements, for example on an artificial cornea. In order to finally ascertain the spatial pulse distance from the laser pulse effect diameter, an overlap factor can be preset, which indicates to what extent adjacent laser pulses are to intersect. Herein, the overlap factor can preset an overlap of adjacent laser pulses, which are on a common or identical laser pulse path, and/or the overlap factor can preset an overlap of laser pulses, which are on adjacent laser pulse paths.

By the invention, the advantage arises that according to used laser pulse energy, the spatial pulse distance optimized therefor can be adjusted in automated manner.

The invention also includes embodiments, by which additional advantages arise.

In an embodiment, the laser pulse effect diameter is determined by d=K*(E_{Pulse}−LIOBₜₕ)\hat{ }(⅓), wherein d is the laser pulse effect diameter, K is the tissue factor, E_{Pulse} is a laser pulse energy and LIOBₜₕ is the laser-induced optical breakthrough threshold. In other words, the laser pulse effect diameter can be determined from the cubic root of the respective energy portion, which is above the optical breakthrough threshold, multiplied by a tissue factor, which can in particular be predetermined for the cornea. A cavitation bubble diameter or for example an area, in which a characteristic change, in particular a laser-induced refractive index change, occurs, is meant by the laser pulse effect diameter. Therein, the laser pulse energy Eₚᵤₗₛₑ can be provided in a range from 10 nJ to 1 μJ, for example 30 nJ to 300 nJ, the laser-induced optical breakthrough threshold LIOBₜₕ can for example be in a range from 10 nJ to 500 nJ, in particular 20 nJ to 100 nJ, and the laser pulse effect diameter can have diameter in a range from 0.35 μm to 50 μm, for example 1 μm to 10 μm.

In a further embodiment, the spatial pulse distance includes a distance between adjacent laser pulses on a laser pulse path. In other words, the spatial pulse distance, which is optimized by the method, can include a distance between subsequent laser pulses, which are on the same laser pulse path. Therein, the pulse distance can be defined as the distance between respective centers of the laser pulses.

In a further embodiment, the distance between adjacent laser pulses is determined by the ascertained laser pulse effect diameter divided by a first overlap factor. Thus, it can be preset by the overlap factor, to what extent the adjacent laser pulses are to intersect. This can be performed by division of the laser pulse effect diameter by the first overlap factor or multiplication by a reciprocal value, wherein the first overlap factor can have a value from 1 to 10, for example from 1 to 3. Furthermore, a first offset term between adjacent laser pulses can additionally be preset, which provides an absolute distance between the laser pulses. For example, the first offset term can have a value of 0 to 10 μm, in particular 0 to 2 μm.

In a further embodiment, the spatial pulse distance includes a distance between adjacent laser pulse paths. Thus, multiple laser pulse paths can for example be provided to generate a laser pulse pattern and thereby to separate a volume body. For example, the laser pulse pattern can be generated in the form of concentric circles, spiral paths, parallel paths, a meandering path and/or further space-filling paths in the cornea, wherein the distance of these paths and thereby of the laser pulses on the paths is adjusted by the method. The spatial pulse distance can thus include the distance of adjacent laser pulses and/or the distance of adjacent laser pulse paths.

In a further embodiment, the distance between adjacent laser pulse paths is determined by the ascertained laser pulse effect diameter divided by a second overlap factor. Thus, it can be preset by the second overlap factor, to what extent the laser pulses of adjacent laser pulse paths are to intersect. This can be performed by division of the laser pulse effect diameter by the second overlap factor or multiplication by a reciprocal value, wherein the second overlap factor can have a value of 1 to 10, for example of 1 to 3. Furthermore, a second offset term between adjacent laser pulse paths can be additionally preset, which provides an absolute distance between the laser pulse paths. The second offset term can for example have a value of 0 to 10 μm, in particular 0 to 2 μm.

In a further embodiment, the spatial pulse distance includes the distance between adjacent laser pulses on a laser pulse path and the distance of adjacent laser pulse paths, wherein a ratio of these distances is set in a range between 0.1 and 10, for example in a range between 0.2 and 5. In other words, preset limit values are provided, which set the relation of the distances of adjacent laser pulses divided by the distances of adjacent laser pulse paths. This means, it is to apply:

$${R}_{1} > {A}_{Laser pulse distance} / {A}_{Laser pulse path distance} < {R}_{2} ,$$

wherein R₁ is a lower limit value, in particular 0.1 or 0.2, A_{Laser pulse distance} is the distance of adjacent laser pulses on a laser pulse path, A_{Laser pulse path distance} is the distance of adjacent laser pulse paths and R₂ is an upper limit value, in particular 10 or 5. Thus, suitable pulse distances can be provided, which are advantageous for a treatment of a cornea. Preferably, the limit values R₁ and R₂ can be automatically adapted depending on a used laser pulse energy to limit the ratio of the distances.

In a further embodiment, the optical breakthrough threshold is measured. In other words, the optical breakthrough threshold can be determined by measurements, in particular on an adequate material, which simulates a cornea, such as for example an artificial cornea and/or an animal cornea. Accordingly, the ascertained optical breakthrough threshold can then be preset to the control device. Thus, an advantageous configuration for determining the optical breakthrough threshold can be provided, and based on the determined optical breakthrough threshold, the laser pulse distances can be determined.

In a further embodiment the optical breakthrough threshold is calculated by the control device. Hereto, the formula

$${LIOB}_{th} = C * \sqrt[3]{τ} * λ * \sqrt{m} * M {2}^{2} / ( {NA}^{2} * SR )$$

can be used for the calculation of the breakthrough threshold of the laser-induced optical breakthrough (LIOBₜₕ), wherein τ is the pulse length of the laser pulses, m is the number of photons, M2 is the quality factor of the laser beam, SR is the Strehl ratio, which indicates a quality factor of the optical beam path, λ is the wavelength and NA is the numerical aperture. C is a proportionality constant for the laser-induced optical breakthrough, which can, for example, be calculated or determined by measurement. Accordingly, the advantage arises that the optical breakthrough threshold can be quickly determined for different adjustments of the ophthalmological laser without having to perform prolonged and expensive measurements.

A further embodiment relates to a method for controlling a treatment apparatus. Therein, the method includes the method steps of at least one embodiment of the method as it was previously described. Furthermore, the method includes controlling the treatment apparatus, wherein the treatment apparatus and/or the ophthalmological laser can be controlled by the optimized spatial pulse distance. Accordingly, control data can for example be provided for the treatment apparatus, which includes a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser can be included in the control data.

The respective method can include at least one additional step, which is executed if and only if an application case or an application situation occurs. For example, the step can include the output of an error message and/or the output of a request for inputting a user feedback. Additionally or alternatively, it can be provided that a default setting and/or a predetermined initial state are adjusted.

A further aspect of the invention relates to a control device, which is formed to perform the steps of at least one embodiment of the previously described method. The control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by the computing unit, whereby the control device is caused to execute the respective embodiment. The control device can be formed as a control chip or control unit. The control device can for example be encompassed by a computer or computer cluster.

A further aspect of the invention relates to a treatment apparatus with at least one eye surgical or ophthalmological laser and a control device, which is formed to perform the steps of at least one embodiment of the previously described method. The respective laser can be formed to perform respective incisions in a cornea by optical breakthrough, in particular to at least partially separate a predefined corneal volume with predefined interfaces of a human or animal eye by optical breakthrough, in particular to at least partially separate it by photodisruption and/or to ablate corneal layers by (photo)ablation and/or to effect a laser-induced refractive index change in the cornea and/or the eye lens.

A further aspect of the invention relates to a computer program. The computer program includes commands, which for example form a program code. The program code can include at least one control dataset with the respective control data for the respective laser. Upon execution of the program code by a computer or a computer cluster, it is caused to execute the previously described method or at least one embodiment thereof.

A further aspect of the invention relates to a computer-readable medium (storage medium), on which the above mentioned computer program and the commands thereof, respectively, are stored. For executing the computer program, a computer or a computer cluster can access the computer-readable medium and read out the content thereof. The storage medium is for example formed as a data memory, in particular at least partially as a volatile or a non-volatile data memory. A non-volatile (e.g., non-transitory) data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the embodiments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 depicts a schematic representation of a treatment apparatus according to an exemplary embodiment;

FIG. 2 depicts an exemplary representation of laser pulses on laser pulse paths for generating incision surfaces.

In the figures, identical or functionally identical elements are provided with the same reference characters.

### DETAILED DESCRIPTION

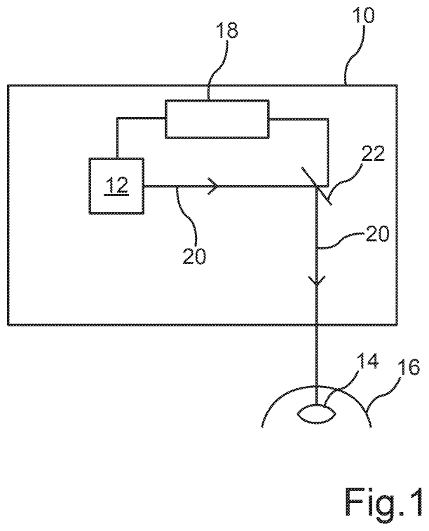

FIG. 1 shows a schematic representation of a treatment apparatus 10 with an ophthalmological laser 12 for the removal of a tissue 14 from a cornea 16 of a human or animal eye by photodisruption and/or photoablation or for a laser-induced refractive index change. For example, the tissue 14 can represent a lenticule and/or volume body, which is to be separated out of the cornea 16 of the eye by the ophthalmological laser 12 for correcting a visual disorder. A geometry of the tissue 14 to be removed can be provided by a control device 18, in particular in the form of control data, such that the laser 12 emits pulsed laser pulses in a pattern predefined by the control data into the cornea 16 of the eye to remove the tissue 14. Alternatively, the control device 18 can be a control device 18 external with respect to the treatment apparatus 10.

Furthermore, FIG. 1 shows that the laser beam 20 generated by the laser 12 can be deflected towards the eye by a beam deflection means 22, namely a beam deflection device such as for example a rotation scanner, to remove the tissue 14. The beam deflection device 22 can also be controlled by the control device 18.

The illustrated laser 12 can preferably be a photodisruptive and/or photoablative laser, which is formed to emit laser pulses in a wavelength range between 300 nanometers and 1400 nanometers, for example between 700 nanometers and 1200 nanometers, at a respective pulse duration between 1 femtosecond and 1 nanosecond, for example between 10 femtoseconds and 10 picoseconds, and a repetition frequency of greater than 10 kilohertz, for example between 100 kilohertz and 100 megahertz. Optionally, the control device 18 additionally comprises a storage device (not illustrated) for at least temporary storage of at least one control dataset, wherein the control dataset or datasets include(s) control data for positioning and/or for focusing individual laser pulses in the cornea.

Furthermore, the control device 18 can be formed to ascertain or determine an optimized spatial pulse distance of laser pulses for treating the cornea 16, in particular for separating the tissue 14. That is, the control device 18 can adjust a spatial distance of laser pulses in automated manner depending on a used laser pulse energy, wherein a distance is preferably adjusted, which is optimized for the laser pulse energy. For this purpose, an optical breakthrough threshold of the cornea 16 can be preset and/or provided to the control device 18. For example, the breakthrough threshold of the cornea 16 can be determined by measurements on materials, which have similar characteristics as the cornea 16. Alternatively, the optical breakthrough threshold or the threshold value of the laser-induced optical breakthrough can be calculated by the control device 18 for the treatment apparatus 10 with the formula:

$${LIOB}_{th} = C * \sqrt[3]{τ} * λ * \sqrt{m} * M {2}^{2} / ( {NA}^{2} * S R )$$

wherein LIOBₜₕ is the optical breakthrough threshold, τ is the pulse length, λ is the wavelength, m is the number of photons, M2 is the quality factor of the laser beam, SR is the Strehl ratio, NA is the numerical aperture and C is a proportionality constant.

According to used laser pulse energy, the control device 18 can then ascertain which laser pulse energy portion is above the optical breakthrough threshold and can calculate a laser pulse effect diameter together with a predetermined tissue factor of the cornea 16. Therein, the laser pulse effect diameter is the diameter of the effect of the respective laser pulse in the cornea 16. Thus, the laser pulse effect diameter can for example be a cavitation bubble diameter or a diameter of an area in which a characteristic change occurs. The laser effect diameter can be calculated by the formula

$$d = K * {( {E}_{Pulse} - {LIOB}_{th} )}^{∧} ( 1 / 3 )$$

wherein d is the respective laser pulse effect diameter, K is the tissue factor, E_{Pulse} is the used laser pulse energy and LIOBₜₕ is the optical breakthrough threshold. Therein, the tissue factor K can for example be predetermined by preceding measurements on corneal tissue. By this formula, it becomes clear that the laser pulse effect diameter continuously increases with the laser pulse energy portion, which is above the optical breakthrough threshold, wherein an optimized laser pulse distance for a treatment of the cornea 16 can thus be ascertained by the control device 18. In particular, a laser pulse distance with known laser pulse effect diameter can be adapted such that the laser pulses generate a contiguous incision in the cornea 16.

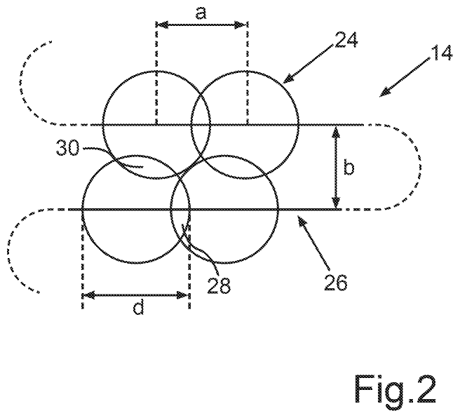

In FIG. 2, an exemplary representation of laser pulses 24 on laser pulse paths 26, for example for generating incision surfaces for removing the tissue 14, is illustrated. As described above, according to used laser pulse energy, the laser pulse effect diameter d can change, wherein an optimized distance a between adjacent laser pulses 24, which are situated on a laser pulse path 26, can thus be determined by the control device 18. Further, a first overlap factor can be preset, by which an overlap area 28 between adjacent laser pulses 24 can be adjusted and/or set.

Alternatively or additionally, a distance b of laser pulse paths 26 can be ascertained by the control device 18 as a spatial pulse distance, wherein a second overlap factor for an overlap area 30 of laser pulses 24 of adjacent laser pulse paths 26 can be preset here too. Therein, the first and/or the second overlap factor can have a value of 1 to 10, for example of 1 to 3, wherein the respective pulse distance a, b can be calculated by the laser pulse effect diameter divided by the respective overlap factor.

Therein, a ratio between the distance of adjacent laser pulses a and the distance of adjacent laser pulse paths b can be preset such that they range within limit values between 0.1 and 10, for example between 0.2 and 5. In particular, the limit values can be adjusted depending on the used laser pulse energy, whereby the ratio of the distances a/b is automatically limited between predetermined limit values.

The control device 18 can finally control the laser 12 or the treatment apparatus 10 for treating the tissue 14 of the cornea 16, wherein the laser pulses 24 are hereto placed in the cornea 16 such that the optimized spatial pulse distances a, b are complied with.

1. A method for determining an optimized spatial pulse distance of laser pulses for an ophthalmological laser of a treatment apparatus, the method comprising:
determining, by a control device of the treatment apparatus, a laser pulse effect diameter based on a predetermined tissue factor of tissue to be irradiated and a laser pulse energy portion above an optical breakthrough threshold, and
determining the optimized spatial pulse distance based on the determined laser pulse effect diameter and a preset overlap factor for adjacent laser pulses.

2. The method according to claim 1, wherein determining the laser pulse effect diameter includes calculating d=K*(E_{Pulse}−LIOBₜₕ)\hat{ }(⅓), wherein d is the laser pulse effect diameter, K is the tissue factor, E_{Pulse} is a laser pulse energy and LIOBₜₕ is a laser-induced optical breakthrough threshold.

3. The method according to claim 1, wherein the optimized spatial pulse distance includes a distance between adjacent laser pulses on a laser pulse path.

4. The method according to claim 3, further comprising determining the distance between adjacent laser pulses by dividing the determined laser pulse effect diameter by a first overlap factor.

5. The method according to claim 1, wherein the optimized spatial pulse distance includes a distance between adjacent laser pulse paths.

6. The method according to claim 5, further comprising determining the distance between adjacent laser pulse paths by dividing the determined laser pulse effect diameter by a second overlap factor.

7. The method according to claim 1, wherein the optimized spatial pulse distance includes the distance between adjacent laser pulses on a laser pulse path and the distance of adjacent laser pulse paths, wherein a ratio of these distances is set in a range between 0.1 and 10, in particular in a range between 0.2 and 5.

8. The method according to claim 1, further comprising measuring the optical breakthrough threshold.

9. The method according to claim 1, further comprising calculating, by the control device, the optical breakthrough threshold.

10. A control device, which is configured to perform the method according to claim 1.

11. A treatment apparatus with at least one ophthalmological laser for separation of a corneal volume with predefined interfaces of a human or animal eye by optical breakthrough, in particular by photodisruption and/or photoablation, and at least one control device according to claim 10.

12. (canceled)

13. A non-transitory computer-readable medium, on which a computer program according is stored, the computer program including commands, which cause a treatment apparatus to execute the method according to claim 1.
